(19) United States
(12) Patent Application Publication
(10) Pub. No.: US 2012/0203357 A1
(43) Pub. Date: Aug. 9, 2012

(54) URETHRAL ANASTOMOSIS DEVICE

(75) Inventors: David Bleicher, Tel Aviv (IL); Eitan Hod, Zichron Ya'akov (IL); Meron Livneh, Binyamina (IL)

(73) Assignee: I.B.I ISRAEL BIOMEDICAL INNOVATIONS LTD., Caesarea Industrial Park (IL)

(21) Appl. No.: 13/501,677

(22) PCT Filed: Oct. 14, 2010
(86) PCT No.: PCT/IL2010/000838

§ 371 Date: Apr. 12, 2012

Related U.S. Application Data
(60) Provisional application No. 61/251,907, filed on Oct. 15, 2009.

(51) Int. Cl. A61F 2/04

(52) U.S. Cl. 623/23.66

(57) ABSTRACT

Provided is a device for urethral anastomosis, for example, following prostatectomy. The device has a flexible slender shaft including a flexible outer sleeve and a flexible inner sleeve inside a lumen of the outer sleeve. A bladder anchoring system anchors the distal end of the inner sleeve inside a urinary bladder, and a urethral anchoring system anchors the outer sleeve in a urethral stump. A ratchet mechanism allows proximal movement of the inner sleeve inside the outer sleeve while preventing distal movement of the inner sleeve inside the outer sleeve. The device is used to urge the urinary bladder towards the internal termination of the urethra to allow anastomosis of the bladder neck to the urethral stump.

### FIELD OF THE INVENTION

This invention relates to medical device, and more specifically to such devices for performing anastomosis of two body organs.

### BACKGROUND OF THE INVENTION

Prostate cancer is the second most common malignancy in males after cutaneous malignancies and is the second most common cause of cancer death among men in the United States. In a radical prostatectomy, the surgeon removes all or most of the patient's prostate. Because the urethra travels through the prostate, the upper part of the urethra is removed in the surgery. In order to restore proper urinary functions, the bladder and the urethra must be reconnected.

Providing this connection is particularly difficult due to the limited working space and the small size of the urethra. The size of the urethra makes it difficult to accurately suture the wall of the urethra to the bladder neck. Suturing tends to cause several problems in recovery, including necrosis of the sutured tissues, stricture of the urethra that impedes the flow of urine through it, and a urethra-bladder connection that is not fluid-tight. In addition, when suturing the urethra to the bladder, the surgeon can possibly inadvertently pierce the nearby neurovascular bundle which can cause incontinence or impotence. Sutures may also tear the urethra, resulting in further complications.

U.S. Pat. No. 7,708,748 to Weisenburgh, II et a discloses an instrument for use in a procedure to effect anastomosis of a patient's bladder and urethra following a prostatectomy. The device has a tube assembly, and an end effector assembly operably supported by the tube assembly. The end effector assembly includes a positioner assembly and an anchor driver assembly in operable mechanical communication with first and second rods of the tube assembly, so as to be actuable by movement of the first and the second rods with respect to a spine member.

### SUMMARY OF THE INVENTION

In its first aspect, the present invention provides an anastomosis device for anastomosing a urinary bladder to a urethra or urethra stump, for example, following prostatectomy. The device comprises a flexible inner sleeve located inside a flexible outer sleeve. A ratchet mechanism allows the inner sleeve to move in a proximal direction inside the outer sleeve while preventing distal sliding of the inner sleeve in the outer sleeve. At the distal end of the inner sleeve is a bladder anchoring system which may comprise a bladder balloon configured to be inflated in a urinary bladder. At the distal tip of the outer shaft is a urethral anchoring system. The urethral anchoring system can be deployed at the internal termination of the urethral stump to anchor the distal end of the outer sleeve inside the urethral stump while permitting drainage of urine through the lumen of the inner sleeve. In one embodiment, the urethral anchoring system comprises a cylindrical array of tines located inside the outer. Inflation of a tines balloon located interiorly to the cylindrical array of tines causes the tines to splay outwardly in which configuration, the tips of the tines become embedded in the urethra to anchor the outer sleeve inside the urethral stump

In its second aspect, the invention provides a method of anastomosing a urethral stump to a urinary bladder, for example following radical prostatectomy, using the anastomosis device of the invention. In accordance with this aspect of the invention, the distal end of the anastomosis device is inserted through the urinary meatus into the urethral stump and the shaft of the device is moved through the stump until the distal end exits through the distal end of the stump into the intraperitoneal space through the bladder neck and into the urinary bladder. The urinary anchoring system is then deployed to anchor the distal end of the inner sleeve inside the urinary bladder. The urethral anchoring system is then deployed to anchor the distal end of the outer sleeve near the internal termination of the urethral stump. The proximal end of the inner sleeve is then pulled proximally, while the outer sleeve is held in place causing the bladder to be urged towards the internal termination of the urethral stump. As mentioned above distal movement of the inner shaft relative to the outer shaft is prevented by the ratchet mechanism. The bladder neck is thus held juxtaposed to the internal termination of the urethral stump. The device remains indwelling until anastomosis of the bladder neck to the urethral stump is complete. After biological anastomosis is achieved between the urethral stump and the bladder neck, the anchoring systems are undeployed and the device is removed from the body.

Thus, in its first aspect, the present invention provides a urethral anastomosis device having a proximal end and a distal end and comprising:

(a) A flexible slender shaft comprising a flexible outer sleeve and a flexible inner sleeve inside a lumen of the outer sleeve;

(b) A bladder anchoring system configured to anchor the distal end of the inner sleeve inside a urinary bladder;

(c) A urethral anchoring system configured to anchor the outer sleeve in a urethral stump and further configured to allow urine to pass through the urethral anchoring system; and

(d) a ratchet mechanism allowing proximal movement of the inner sleeve inside the outer sleeve while preventing distal movement of the inner sleeve inside the outer sleeve.

The bladder anchoring system may comprise a bladder balloon configured to be inflated in the urinary bladder. The balloon may have an ellipsoid shape when inflated.

The ratchet mechanism of the device may comprise:

(a) a flexible cylinder consisting of an array of circular asymmetric ratchet teeth surrounding the inner sleeve; and

(b) a rigid cylinder attached to an inner surface of the outer sleeve and having a pawl configured to engage the ratchet teeth of the flexible cylinder.

The urethral anchoring system of the device may comprise:

(a) a cylindrical array of pointed tines surrounding the outer sleeve, the tines having an undeployed configuration in which the tines are in an undeployed position parallel to an outer surface of the outer sleeve and a deployed position in which the tines are splayed outward from the surface of the outer sleeve; and

(b) a tines balloon located inside the cylindrical array of tines, inflation of the tines balloon causing the tines to move from the undeployed position of the tines to the deployed position of the tines.

The urethral anchoring system may further comprise a collapsing sleeve surrounding the cylindrical array of tines, the collapsing sleeve causing the tines to move from the deployed configuration of the tines to the undeployed configuration of the tines when the tines balloon is not inflated. Each tine may be provided with a hinge and each tine may have a point including a shoulder. The tines balloon may have a cylindrical shape.

The device of the invention may further comprise a funnel assembly at the proximal end of the device comprising:

(a) a first opening confluent with a lumen of the inner sleeve configured to drain urine from the urinary bladder after deployment of the device in a urinary tract;

(b) a second opening confluent with a longitudinal passageway in the wall of the inner sleeve for delivering an inflation medium, to an interior of the bladder balloon in order to inflate the bladder balloon; and

(c) a third opening confluent with a passageway in the wall of the outer shaft for delivering an inflation medium to the tines balloon in order to inflate the tines balloon.

In its second aspect, the invention provides method for urethral anastomosis between a urinary bladder neck and a urethral stump comprising:

(a) inserting the distal end of the device according to any one of the previous claims through a urinary meatus into the urethral stump until the distal end of the device is located inside the bladder;

(b) anchoring the distal end of the device inside the urinary bladder by the bladder anchoring system;

(c) sliding the inner sleeve of the device proximally inside the outer sleeve until the bladder neck is in contact with an internal termination of the urethral stump; and

(d) anchoring the distal end of the outer sleeve in the urinary stump by the urethral anchoring system.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1a shows a side view of a urethral anastomosis device in accordance with one embodiment of the invention, FIG. 1b shows the device of FIG. 1a in a perspective view, and FIG. 1c shows the funnel assembly of the device of FIG. 1a;

FIG. 2a shows the urethral anchoring system of the device of FIG. 1 in an undeployed configuration and FIG. 2b shows the urethral anchoring system in a deployed configuration;

FIG. 3 shows a cross section of the outer sleeve of the device of FIG. 1;

FIG. 4 shows the bladder balloon of the device of FIG. 1;

FIG. 5 shows the ratchet sleeve of the device of FIG. 1;

FIG. 6 shows the cylinder comprising a pawl of the device of FIG. 1;

FIG. 7a shows an array of tines for use in the device of FIG. 1, FIG. 7b shows the array of tines rolled into a cylinder, and FIG. 7c shows an enlargement of the tine tip;

FIG. 8 shows the tines balloon of the device of FIG. 1,

FIG. 9 shows a cross section of the inner sleeve of the device of FIG. 1; and

FIG. 10a shows the male anatomy after a radical prostatectomy, FIG. 10b shows the device of FIG. 1 after insertion of the distal end of the device into a urinary bladder prior to inflation of the bladder balloon, FIG. 10c shows the device of FIG. 1 after insertion of the distal end of the device into a urinary bladder after inflation of the bladder balloon, and after deployment the urethral anchoring system in the urethral stump, and FIG. 10d shows juxtaposition of the urinary bladder to the internal termination of the urethral stump.

### DETAILED DESCRIPTION OF EMBODIMENTS

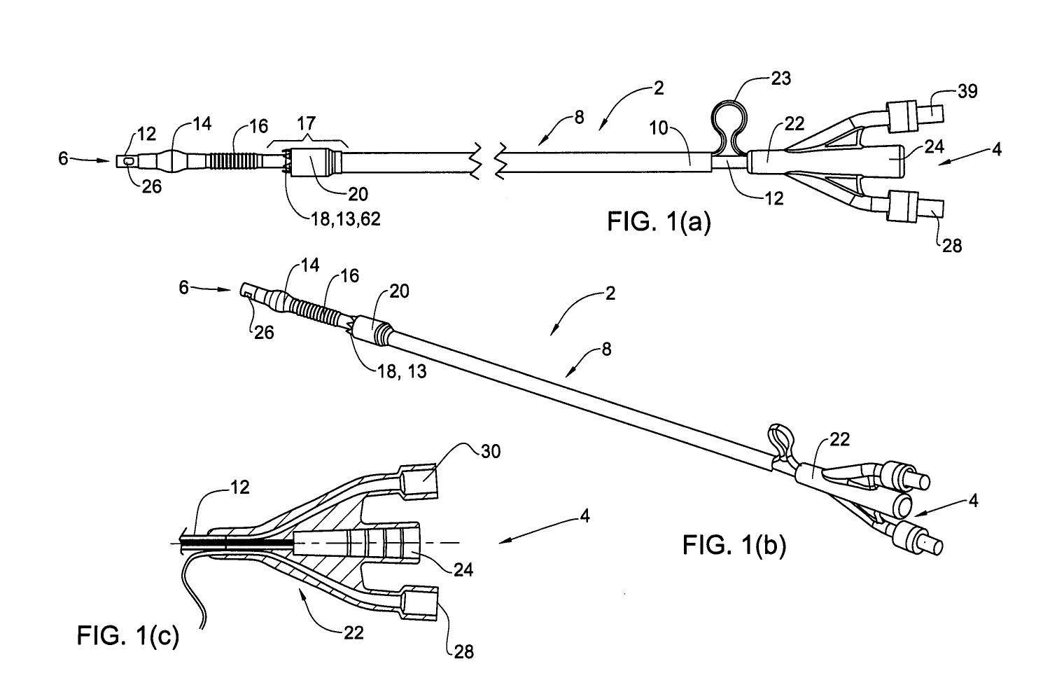

FIG. 1 shows an anastomosis device 2 in accordance with one embodiment of the invention. The device 2 is shown in side view in FIG. 1a and in perspective view in FIG. 1b. The device 2 has a proximal end 4 and a distal end 6. A slender flexible shaft 8 extends between the proximal and distal ends. The shaft includes a flexible outer sleeve 10 and a flexible inner sleeve 12. The inner and outer sleeves may be made, for example, from silicone rubber or PVC. The inner sleeve 12 is coaxial with the outer sleeve 10 and is slidable in the lumen of the outer sleeve, by pulling back on the proximal end of the inner sleeve 12 while immobilizing the proximal end of the outer sleeve. The inner surface of the outer sleeve and the outer surface of the inner sleeve may be lubricated, for example, with Parelene, in order to facilitate relative movement of the two sleeves.

At the distal end of the inner sleeve 12 is a bladder anchoring system which in this embodiment comprises a bladder balloon 14, shown in detail in FIG. 4a. The bladder balloon 14 is configured to be inflated in a urinary bladder, as explained below. The bladder balloon has two symmetric openings 32 and a central bulge 34. The openings 32 are dimensioned to allow the bladder balloon 14 to be mounted snugly onto the distal end of the inner sleeve 12. After mounting the bladder balloon onto the distal end of the inner sleeve, as shown in FIG. 1, the openings 32 are glued or welded to the outer surface of the inner sleeve. FIG. 4b shows a longitudinal section of the bladder balloon. The bladder balloon wall thickness decreases progressively from the middle of the balloon towards the openings 32 of the balloon. This tends to cause the bladder balloon to assume an ellipsoidal shape when inflated rather than a spherical shape. Each leg of the bladder balloon contains a cylindrical recess 36 to accommodate glue without affecting the outer diameter of the balloon. The balloon might be in an inverse shape balloon.

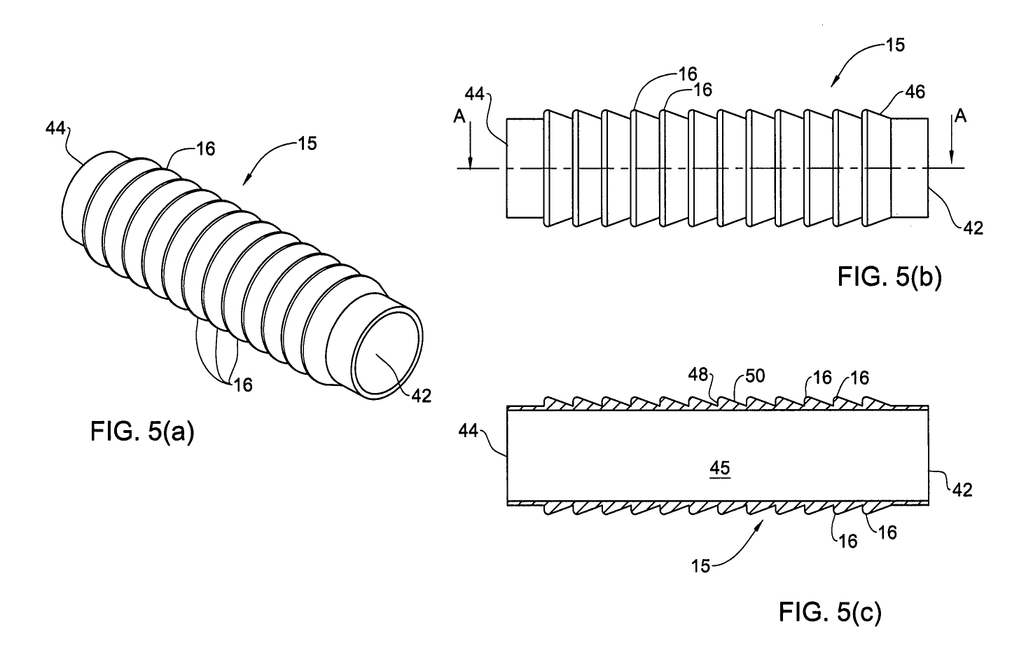

Proximal to the bladder balloon 14, a cylinder 16 consisting of an array of circular ratchet teeth 46 is over molded onto the inner sleeve 12. The cylinder 15 is shown in greater detail in FIG. 5. The cylinder 15 is shown in a perspective view in FIG. 5a, in a side view in FIG. 5b, and in a longitudinal sectional view in FIG. 5c. The cylinder 15 is made from a flexible material 15, such as silicone rubber, and has a distal end 44 and a proximal end 42. A lumen 45 of the cylinder is adapted to receive the inner sleeve. Each ratchet tooth 46 has a distal surface 48 that is annular in shape. Each tooth 46 has a proximal surface 50 that is conical in shape.

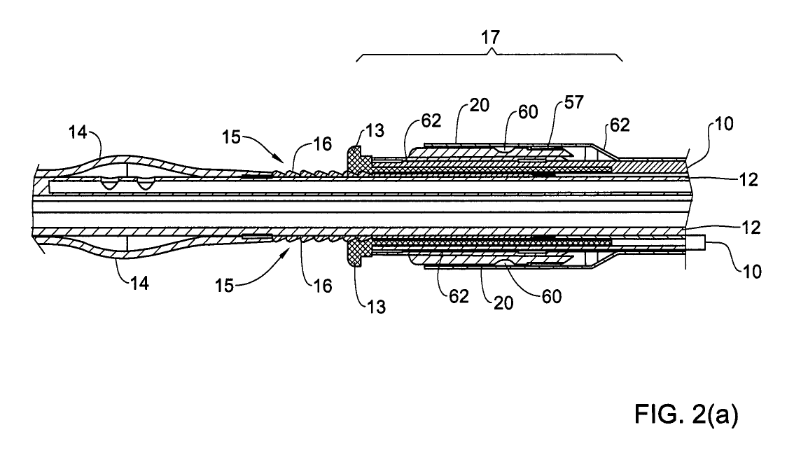

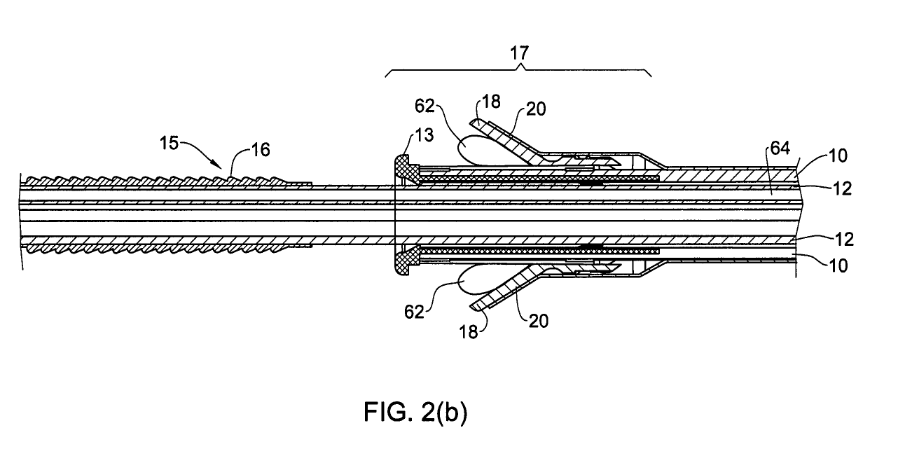

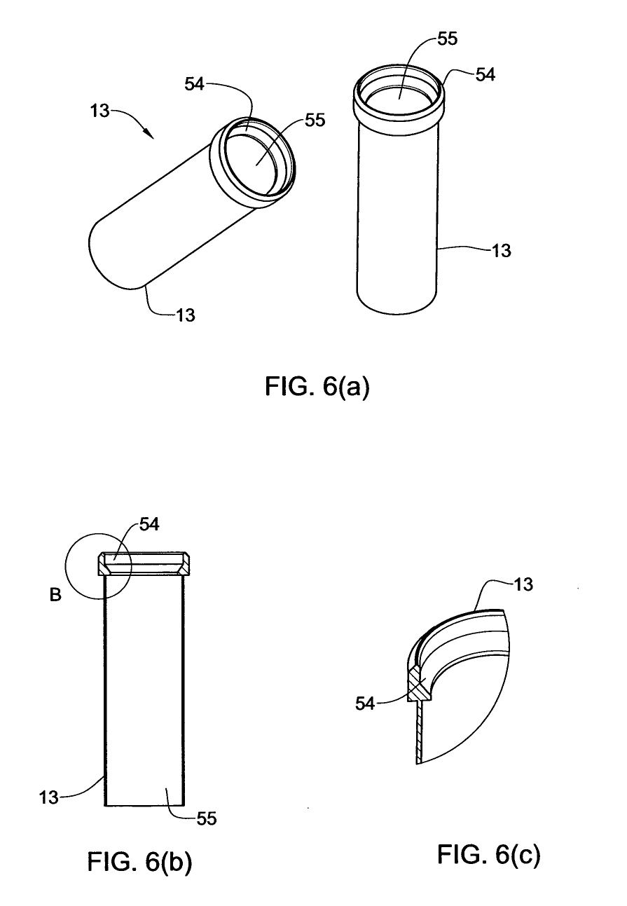

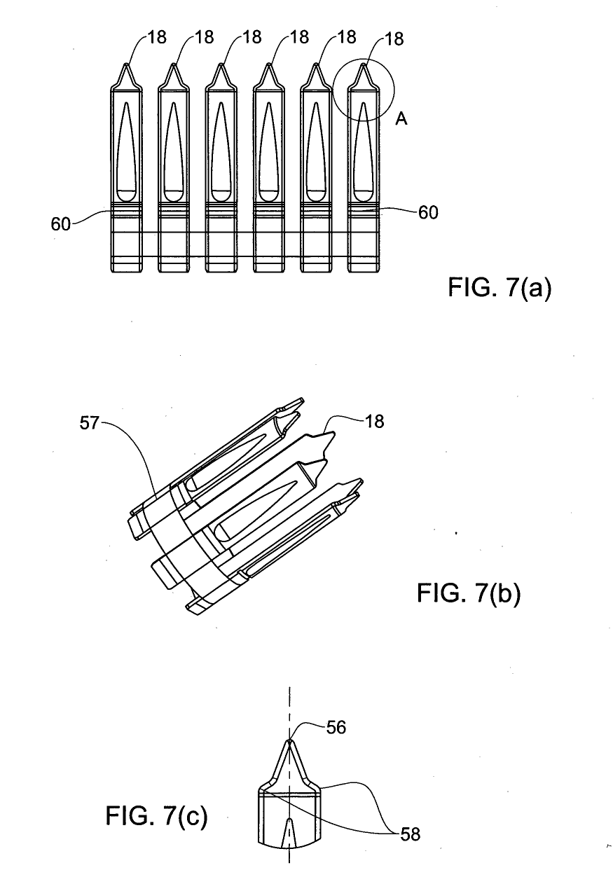

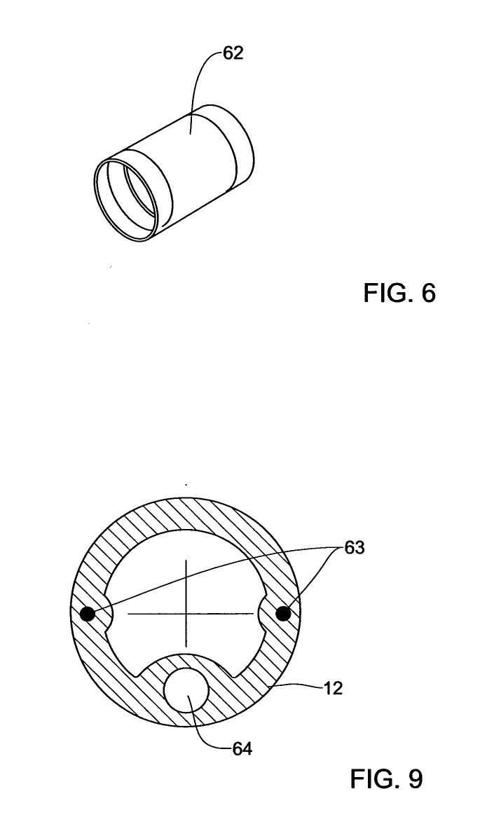

At the distal tip of the outer shaft 10, is a urethral anchoring system 17, shown in greater detail in FIG. 2. The anchoring system 17 comprises a cylinder 13 that is mounted onto the inner surface of the outer sleeve 10, and is shown in greater detail in FIG. 6. The cylinder 13 is shown in two perspective views in FIG. 6a, and in a longitudinal sectional view in FIG. 6b. The cylinder 13 is made from a rigid material such as metal or plastic. At the distal end of the cylinder 13 is a circular pawl 54, shown in greater detail in FIG. 6c. The pawl 54 faces a lumen 55 of the cylinder and engages the ratchet teeth 16 on the inner sleeve 12. Due to the flexibility of the ratchet sleeve teeth and the asymmetric profile of the teeth 46, the ratchet sleeve 16 can move roximally through the pawl 54, but is prevented from moving distally by the pawl. Since the ratchet sleeve 16 is affixed onto the outer surface of the inner sleeve 12 while the ratchet tip 13 is affixed to the inner surface of the outer sleeve 10, the ratchet engagement between the ratchet sleeve and the ratchet tip allows the inner sleeve 12 to move proximally inside the outer sleeve while preventing distal movement of the inner sleeve 12 relative to the outer sleeve 10.

The urethral anchoring system 17 further comprises a cylindrical array of tines 18 that is located inside the outer sleeve 10 and exterior to the cylinder 13. Formation of the array of tines 18 is shown in FIG. 7. A planar sheet of tines 18 is first formed (FIG. 7a). The sheet of tines may be made, for example, from plastic or metal and may be made by injection molding. Each tine has a tip 56 shown enlarged in FIG. 7c. The prong tip is shaped to have a shoulder 58 on each side of the tip 56. The sheet of tines is then bent into a cylindrical shape (FIG. 7b), and is maintained in the cylindrical shape by means of a ring 57. The cylindrical array of tines is positioned in the anchoring system 17 with the tips pointing distally. Each tine 18 has an integral hinge 60.

The urethral anchoring system further comprises a tines balloon 62, shown in FIG. 8 in greater detail. The tines balloon 62 is a cylindrical balloon located interiorly to the cylindrical array of tines. Inflation of the tines balloon causes the tines to splay outwardly, as shown in FIG. 2a which brings the urethral anchoring system 17 from its undeployed configuration shown in FIG. 2a to its deployed configuration shown in FIG. 2b. As explained below, in the deployed configuration, the tips of the tines become embedded in the urethra to anchor the outer sleeve 10 inside the urethral stump. The shoulder determines the depth to which the tine points can penetrate into the mucosa of the urethral.

The tines balloon 62 is of some cylindrical shape and is dimensioned to be mounted onto the inner shaft 12 and to be received in the interior of the cylindrical array of tines 18. The wall thickness of the tines balloon decreases towards the distal end of the balloon 65. When the tines balloon is inflated, it adopts a conical shape, causing the distal diameter to be larger than the proximal diameter. The each tine bends outward at its integral hinge 60 causing the array of tines to adopt a deployed configuration in which the tines splay outward pointing distally giving the array of tines a conical shape. When it is desired to bring the tines 18 from the deployed configuration back to the undeployed configuration, a re-collapsing sleeve 20 (FIG. 1) mounted on the outer shaft 10 forces the tines to bend at the hinge 60 back to the undeployed configuration when the tines balloon 62 is not inflated.

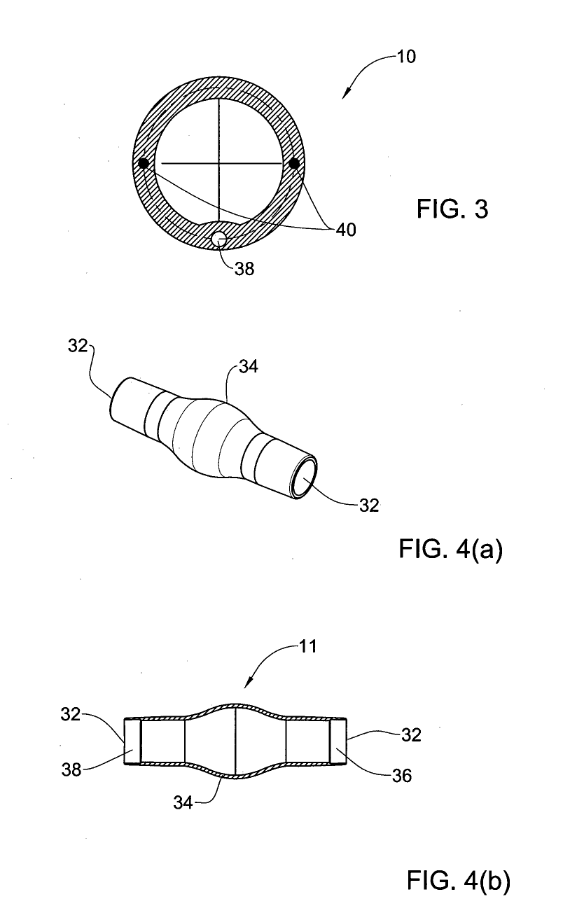

Referring again to FIG. 1, at the proximal end of the inner shaft 12 is a funnel assembly 22, shown in a longitudinal sectional view in FIG. 1c. The funnel assembly 22 has three openings. An opening 24 of the funnel assembly is confluent with the lumen of the inner sleeve 12 and serves to drain urine from the urinary bladder after deployment of the device in a urinary tract. Urine can flow through the lumen of the inner sleeve when the bladder anchoring system is undeployed as well as when the bladder anchoring system is deployed. Urine enters an aperture 26 at the distal end of the inner sleeve, flows through the lumen of the inner sleeve 12 and is released through the opening 24. The opening 24 is typically connected to a urine collection bag (not shown). A second opening 28 of the funnel assembly is confluent with a longitudinal passageway 64 (see FIG. 9) in the wall of the inner sleeve 12 for delivering an inflation medium, such as air or saline, to the interior of the bladder balloon 14 in order to inflate the bladder balloon. A single direction flow valve is assembled to control the flow through the opening 28. A third opening 30 is confluent with a passageway 38 (see FIG. 3) in the wall of the outer shaft 10 for delivering an inflation medium to the tines balloon 18 in order to inflate the tines balloon. This opening is controlled by a similar valve to opening 28.

The device may include an ergonomically designed external insertion handle to allow the device to be manipulated in the body under physical feel supervision.

FIG. 9 shows a cross section of the inner sleeve 10. In the wall of the inner shaft is the passageway 64 for delivering the inflation medium to the bladder balloon from the funnel assembly 22, as explained above. Two threads 63, made from a non stretchable material such as cotton or silk, are embedded in the wall of the outer shaft, extending longitudinally along the length of the outer shaft. The threads 63 prevent the stretching of the inner shaft during deployment of the device 2, as explained below.

FIG. 3 shows a cross section of the outer sleeve 10. In the wall of the outer sleeve is the passageway 38 for delivering the inflation medium from the funnel assembly 22 to the tines balloon, as explained above. The passage way 38 is connected to the funnel assembly 22 by a tube 23 (FIG. 1) that initially bulges out from the shaft 8. When the inner sleeve is pulled proximally inside the outer sleeve during deployment of the device 2, as explained below, the tube 23 straightens and lies parallel to the inner sleeve. Two wires 40 are embedded in the wall of the outer sleeve 10, extending longitudinally along the length of the outer sleeve. The wires 40 prevent longitudinal constriction of the outer shaft during deployment of the device 2, as explained below.

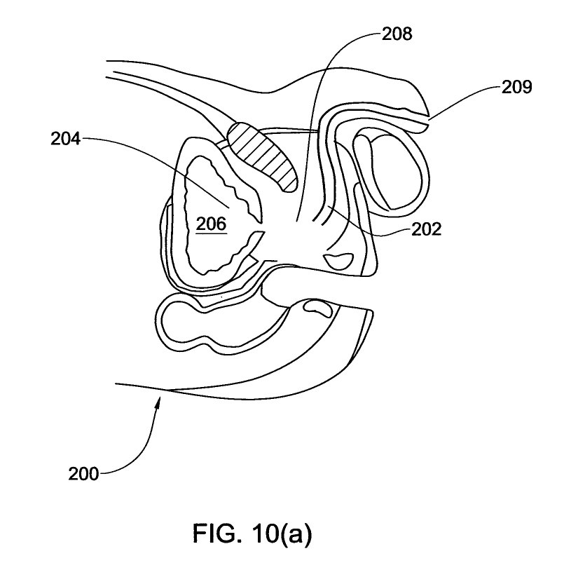

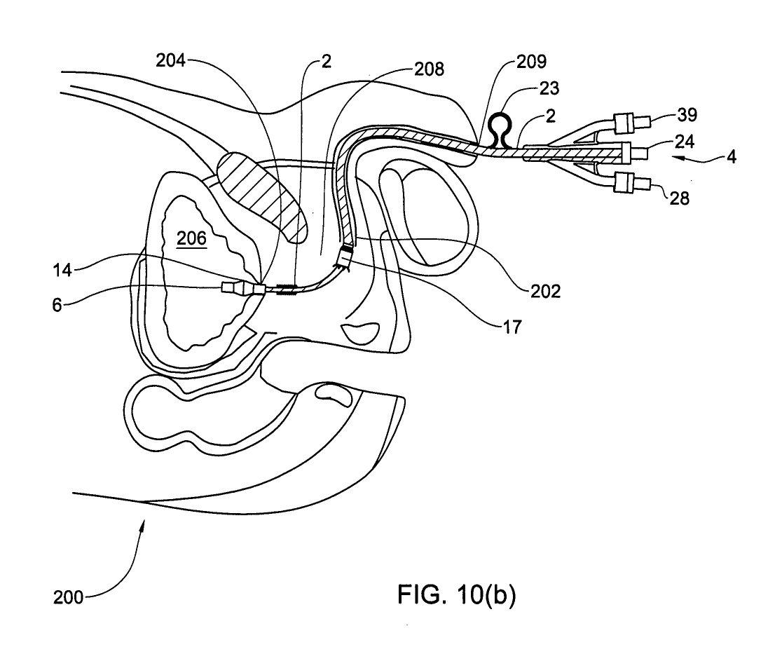

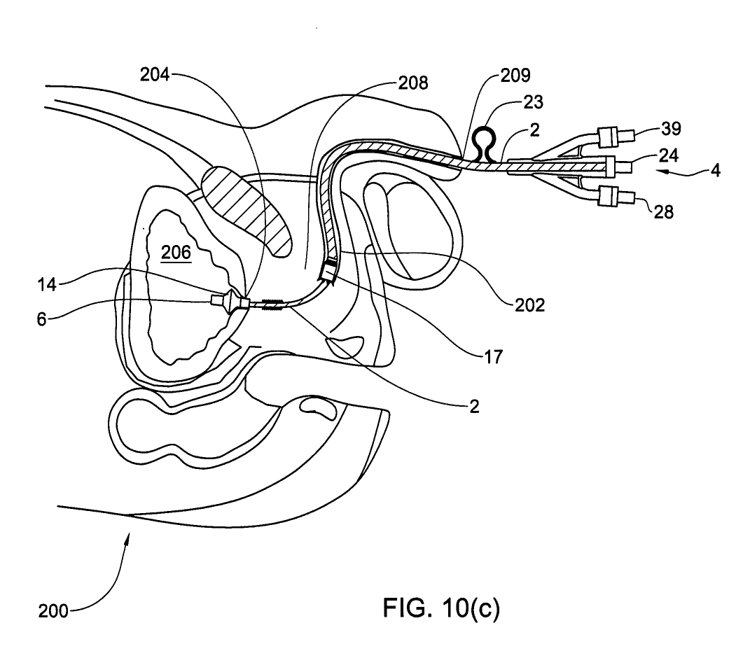

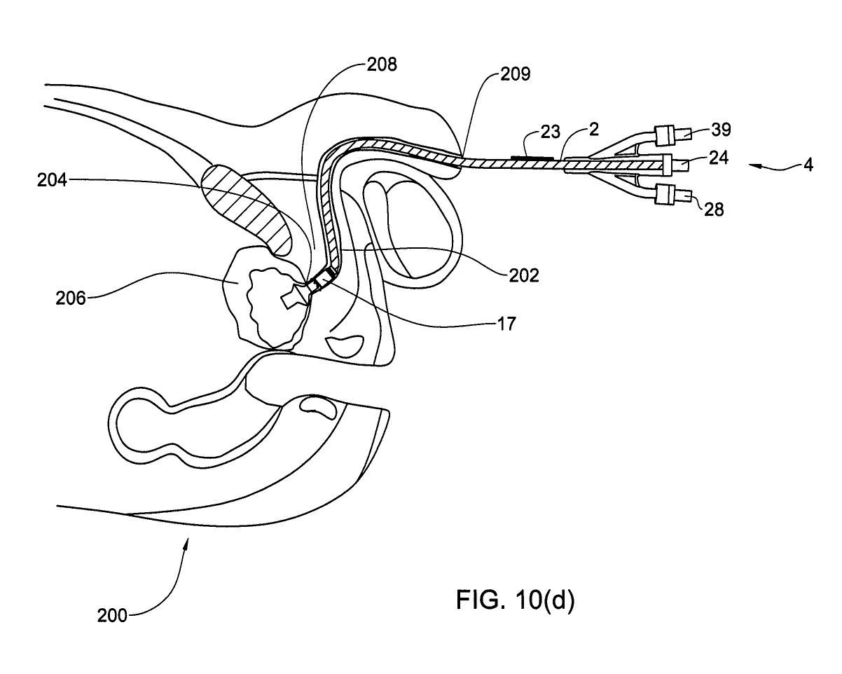

FIG. 10 shows use of the device 2 for performing urethral anastomosis in a male subject 200. Referring first to FIG. 10a, following prostatectomy, a urethral stump 202 is present that terminates internally at a distance from the neck 204 to the urinary bladder 206. As shown in FIG. 10b, in use, the distal end 6 of the device 2 is inserted through the urinary meatus 209 into the urethral stump 202 and the shaft of the device is moved through the stump, until the distal end 6 exits through the distal end of the stump into the intraperitoneal space 208. As the device continues to be moved towards the bladder, the distal end of the device eventually enters the bladder neck 204, and then enters into the urinary bladder 206. Movement of the device inside the body may be monitored laparoscopically or by ultrasound imaging. During insertion of the device, an elastic rod may be inserted into the draining lumen of the inner shaft 12 in order to prevent inappropriate bending and longitudinal compression of the shaft 8. When the insertion has been accomplished, the rod is removed.

The opening 28 of the funnel 22 is now connected to a source of a pressurized fluid such as a syringe (not shown) and the bladder balloon 14 is inflated inside the urinary bladder 206 (FIG. 10c) by delivering the pressurized fluid through the passageway 64. After inflation of the bladder balloon the source of pressurized fluid maybe detached from the opening 28, and the device 2 is then pulled back by the outer sleeve until the tine tips are located 2-3 mm proximal to the internal termination of the urethral stump 202. The bladder is pulled towards the pelvic floor by the bladder balloon as the device is pulled back.

Now the tines balloon is inflated by attaching a source of pressurized fluid such as a syringe (not shown) to the opening 39 and delivering the pressurized fluid through the passageway 38. Inflation of the tines balloon deploys the anchoring system causing the tine tips to splay outwards (FIG. 2b) and to penetrate the urethral mucosa. The presence of the shoulder 58 in the tines tends to prevent the tips from entering the underlying smooth muscle of the urethra. The funnel 22 is then pulled proximally, while the outer sleeve is held in place. The inner sleeve thus slides proximally in the outer sleeve causing the bladder balloon to urge the bladder towards the internal termination of the urethral stump (FIG. 10d). As mentioned above distal movement of the inner shaft 12 relative to the outer shaft 10 is prevented by the ratchet mechanism formed by the ratchet sleeve 16 and the ratchet tip 13. With the tips 56 of the deployed tines 18 embedded in the mucosa of the urethral stump and the bladder balloon inflated in the bladder, the bladder neck is held juxtaposed to the internal termination of the urethral stump. The device 2 remains indwelling until anastomosis of the bladder neck to the urethral stump is complete. The angular opening of the urethral stump which is caused by the deployed position of the tines exposes the urethral mucosa to the bladder mucosa tissue which tends to allow a smooth and scarless anastomosis. After biological anastomosis is achieved between the urethral stump and the bladder neck, the tines balloon is deflated. The collapsing sleeve 20 causes the tines to return to their undeployed configuration (FIG. 2a), and the device 2 can be removed from the body.

In order to prevent local necrosis of the bladder neck by the constant pressure of the bladder balloon during the period that the device is in place, the bladder balloon wall thickness may vary along the diameter of the bladder balloon 14. By this variation, the bladder balloon inflates to a non symmetric shape which prevents necrosis from occurring.

1.-11. (canceled)

12. A urethral anastomosis device having a proximal end and a distal end and comprising:
(a) a flexible slender shaft comprising a flexible outer sleeve and a flexible inner sleeve inside a lumen of the outer sleeve;
(b) a bladder anchoring system configured to anchor the distal end of the inner sleeve inside a urinary bladder;
(c) a urethral anchoring system configured to anchor the outer sleeve in a urethral stump and further configured to allow urine to pass through the urethral anchoring system; and
(d) a ratchet mechanism allowing proximal movement of the inner sleeve inside the outer sleeve while preventing distal movement of the inner sleeve inside the outer sleeve.

13. The device according to claim 12, wherein the bladder anchoring system comprises balloon configured to be inflated in the urinary bladder.

14. The device according to claim 13, wherein the balloon has an ellipsoid shape when inflated.

15. The device according to claim 12, wherein the ratchet mechanism comprises:
(a) a flexible cylinder consisting of an array of circular asymmetric ratchet teeth surrounding the inner sleeve; and
(b) a rigid cylinder attached to an inner surface of the outer sleeve and having a pawl configured to engage the ratchet teeth of the flexible cylinder.

16. The device according to clam 12, wherein the urethral anchoring system comprises:
(a) a cylindrical array of pointed tines surrounding the outer sleeve, the tines having an undeployed configuration in which the tines are in an undeployed position parallel to an outer surface of the outer sleeve and a deployed position in which the tines are splayed outward from the surface of the outer sleeve; and
(b) a tines balloon located inside the cylindrical array of tines, inflation of the tines balloon causing the tines to move from the undeployed position of the tines to the deployed position of the tines.

17. The device according to claim 16, wherein the urethral anchoring system further comprises a collapsing sleeve surrounding the cylindrical array of tines, the collapsing sleeve causing the tines to move from the deployed configuration of the tines to the undeployed configuration of the tines when the tines balloon is not inflated.

18. The device according to claim 16, wherein each tine is provided with a hinge.

19. The device according to claim 16, wherein each tine has a point including a shoulder.

20. The device according to claim 16, wherein the tines balloon has a cylindrical shape.

21. The device according to claim 12, further comprising a funnel assembly at the proximal end of the device comprising:
(a) a first opening confluent with a lumen of the inner sleeve configured to drain urine from the urinary bladder after deployment of the device in a urinary tract;
(b) a second opening confluent with a longitudinal passageway in the wall of the inner sleeve for delivering an inflation medium, to an interior of the bladder balloon in order to inflate the bladder balloon; and
(c) a third opening confluent with a passageway in the wall of the outer shaft for delivering an inflation medium to the tines balloon in order to inflate the tines balloon.

22. A method for urethral anastomosis between a urinary bladder neck and a urethral stump comprising:
(a) inserting the distal end of the device according to any one of the previous claims through a urinary meatus into the urethral stump until the distal end of the device is located inside the bladder;
(b) anchoring the distal end of the device inside the urinary bladder by the bladder anchoring system;
(c) sliding the inner sleeve of the device proximally inside the outer sleeve until the bladder neck is in contact with an internal termination of the urethral stump; and
(d) anchoring the distal end of the outer sleeve in the urinary stump by the urethral anchoring system.
